(19) United States
(12) Patent Application Publication
(10) Pub. No.: US 2025/0025298 A1
(43) Pub. Date: Jan. 23, 2025

(54) EXPANDABLE BRAIDED INTRODUCER SHEATH

(71) Applicant: St. Jude Medical, Cardiology Division, Inc., St. Paul, MN (US)

(72) Inventors: William H. Peckels, Robbinsdale, MN (US); Tracee Eidenschink, Wayzata, MN (US); Randolf Von Oepen, Aptos, CA (US); Hans Rieckmann, Minneapolis, MN (US)

(73) Assignee: St. Jude Medical, Cardiology Division, Inc., St. Paul, MN (US)

(21) Appl. No.: 18/773,976

(22) Filed: Jul. 16, 2024

Related U.S. Application Data
(60) Provisional application No. 63/514,170, filed on Jul. 18, 2023.

(51) Int. Cl. A61F 2/24; A61M 25/00

(52) U.S. Cl. CPC A61F 2/243; A61F 2210/0014; A61M 25/005; A61M 25/0074

(57) ABSTRACT

In some examples, an introducer sheath extends from a proximal end to a distal end, and includes a hub disposed at the proximal end, and a body coupled to the hub and extending between the proximal end and the distal end, the body defining a lumen and having a collapsed condition and an expanded condition, the body having a braided material and an elastomeric material covering the braided material, the body having a flared distal end.

### CROSS-REFERENCE TO RELATED APPLICATIONS

The present application claims priority to U.S. Provisional Ser. No. 63/514,170, filed Jul. 18, 2023, the disclosure of which is hereby incorporated by reference in its entirety as if fully set forth herein.

### BACKGROUND OF THE DISCLOSURE

The present disclosure is related to percutaneous medical procedures, and more particularly to devices providing access into the body for performing percutaneous medical procedures. Still more particularly, the present disclosure is related to prosthetic heart valve replacement, including devices, systems, and methods for transcatheter delivery of collapsible prosthetic heart valves into a patient.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent and a balloon-expandable stent or braided structure. To place such valves into a delivery apparatus and ultimately into a patient, the valve must first be collapsed or crimped to reduce its circumferential size.

When a collapsed prosthetic valve has reached the desired implant site in the patient (e.g., at or near the annulus of the patient's heart valve that is to be replaced by the prosthetic valve), the prosthetic valve can be deployed or released from the delivery apparatus and re-expanded to full operating size. For balloon-expandable valves, this generally involves deploying the valve, assuring its proper location, and then expanding a balloon positioned within the valve stent. For self-expanding valves, on the other hand, the stent automatically expands as the sheath covering the valve is withdrawn.

Despite the various improvements that have been made to the collapsible prosthetic heart valve delivery process, conventional delivery devices, systems, and methods suffer from some shortcomings. For example, in conventional delivery devices for heart valves, large introducers risk traumatizing the iliac or femoral arteries, and the risk of trauma increases with introducers having larger diameters. Additionally, the large diameters of these devices continue to be a challenge due to the size of the collapsed valve. As an example, the outer diameter of a transcatheter mitral valve replacement device can be in the range of 36 F and the corresponding introducer sheath may be 40 F, requiring a surgical cut down for most cases. The ideal procedure for the interventional cardiologist is a minimal profile access with no vascular complications.

There therefore is a need for further improvements to the devices, systems, and methods for transcatheter delivery of collapsible prosthetic heart valves, and in particular, the introduction of such prosthetic heart valves into the heart. Among other advantages, the present disclosure may address one or more of these needs.

### BRIEF SUMMARY OF THE DISCLOSURE

In some examples, an introducer sheath extends from a proximal end to a distal end, and includes a hub disposed at the proximal end, and a body coupled to the hub and extending between the proximal end and the distal end, the body defining a lumen and having a collapsed condition and an expanded condition, the body having a braided material and an elastomeric material covering the braided material, the body having a flared distal end.

In some examples, an introducer sheath extends from a proximal end to a distal end and includes a hub disposed at the proximal end, and a body having a lumen and extending between the proximal end and the distal end, the body having a collapsed condition and an expanded condition, the body having a double layer of a braided material and an elastomeric material covering the braided material.

### BRIEF DESCRIPTION OF THE DRAWINGS

Various embodiments of the present disclosure will now be described with reference to the appended drawings. It is to be appreciated that these drawings depict only some embodiments of the disclosure and are therefore not to be considered limiting of its scope.

FIG. 1A is a top plan view of a portion of an operating handle of a prior art delivery device for a collapsible prosthetic heart valve, shown with a partial longitudinal cross-section of the distal portion of a catheter assembly;

FIG. 1B is a side elevational view of the handle of FIG. 1A;

FIG. 2 is a side elevational view of a prior art prosthetic heart valve;

FIG. 3 illustrates one example of an introducer sheath folding technique;

FIGS. 4A-C are schematic perspective and cross-sectional views of an expandable introducer sheath having a body comprising a braided material and an elastomeric material;

FIGS. 4D-F are schematic detailed views of a hub, a flared distal end and a tip roll of introducer sheaths according to some embodiments;

FIGS. 4G-H are magnified photographs of a braided material in a collapsed and expanded condition, respectively;

FIGS. 4I-J are schematic models of a braided material in a collapsed and expanded condition, respectively;

FIGS. 5A-E are schematic side and cross-sectional views showing various examples of coupling an elastomeric material to a braided material;

FIG. 6A-E illustrate a flared distal end of a body, and several examples of dilator flared distal end retaining features for shielding or retaining the flared distal end during insertion;

FIGS. 7A-C illustrate steps of using a two-part dilator for retaining the flared distal end of the introducer sheath;

FIGS. 8-9C illustrate steps of using a releasable elastomer and a split sheath for retaining the flared distal end of the introducer sheath;

FIG. 10 is a procedural flow chart showing the use of a locking dilator;

FIGS. 11A-F are photographs showing an exemplary introducer sheath and the relevant measurements of the exemplary introducer sheath during the use of a dilator and the delivery of a prosthetic heart valve;

FIG. 12 is a photograph showing maximum expansion capability of an introducer sheath;

FIGS. 13A-B illustrate the use of buttressing axial wires to strengthen an introducer sheath;

FIGS. 14A-B illustrate the use of stabilizing wires within peripheral lumens to provide axial support to an introducer sheath;

FIG. 15 is a schematic cross-sectional view of an introducer sheath with folded jackets; and

FIGS. 16A-B are photographs showing axial folding of braided material.

### DETAILED DESCRIPTION

As used herein, the terms “proximal,” “distal,” “leading” and “trailing” are to be taken as relative to a user using the disclosed delivery devices. “Proximal” or “trailing end” are to be understood as relatively close to the user, and “distal” or “leading end” are to be understood as relatively farther away from the user. Also, as used herein, the words “substantially,” “approximately,” “generally” and “about” are intended to mean that slight variations from absolute are included within the scope of the structure or process recited.

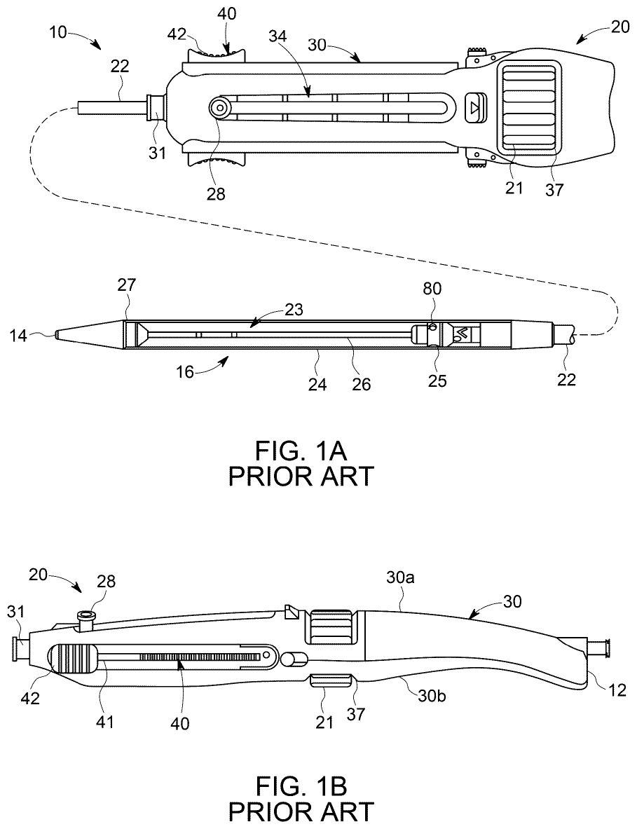

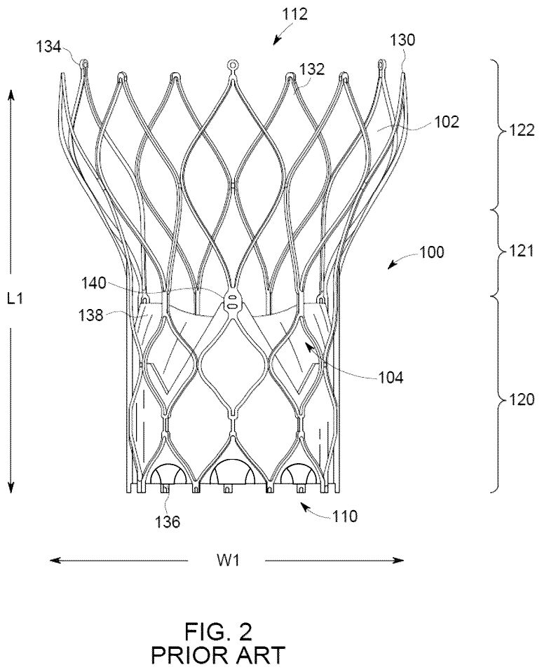

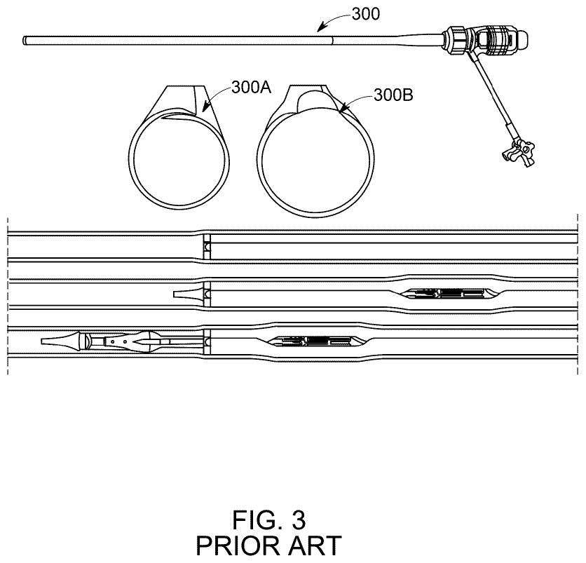

In the description which follows, the structure and function of a transaortic or transfemoral delivery device will be described. It will be understood, however, that the devices and methods disclosed herein also may be used with a transapical or transseptal delivery device. Indeed, the devices and methods described herein may be used in connection with any minimally invasive procedure to provide a passageway for any type of small profile medical device or instrument into a patient's body. An exemplary transaortic delivery device 10 for delivering a prosthetic heart valve into a patient is shown in FIGS. 1A and 1B. Transaortic delivery device 10 has a catheter assembly 16 for delivering the heart valve to and deploying the heart valve at a target location, and an operating handle 20 for controlling deployment of the valve from the catheter assembly. Delivery device 10 extends from a proximal end 12 to an atraumatic tip 14 at the distal end of catheter assembly 16. Catheter assembly 16 is adapted to receive a collapsible prosthetic heart valve (not shown) in a compartment 23 defined around an inner shaft 26 and covered by a distal sheath 24.

Inner shaft 26 may extend from operating handle 20 to atraumatic tip 14 of the delivery device, and may include a retainer 25 affixed thereto at a spaced distance from tip 14 and adapted to hold a collapsible prosthetic valve in compartment 23. Retainer 25 may have recesses 80 therein that are adapted to hold corresponding retention members of the valve. Inner shaft 26 may be made of a flexible material such as braided polyimide or polyetheretherketone (PEEK), for example. Using a material such as PEEK may improve the resistance of inner shaft 26 to kinking while catheter assembly 16 is tracking through the vasculature of a patient.

Distal sheath 24 surrounds inner shaft 26 and is slidable relative to the inner shaft such that it can selectively cover or uncover compartment 23. Distal sheath 24 is affixed at its proximal end to an outer shaft 22, the proximal end of which is connected to operating handle 20 in a manner to be described. Distal end 27 of distal sheath 24 abuts atraumatic tip 14 when the distal sheath is fully covering compartment 23, and is spaced apart from the atraumatic tip when compartment 23 is at least partially uncovered.

Operating handle 20 is adapted to control deployment of a prosthetic valve located in compartment 23 by permitting a user to selectively slide outer shaft 22 proximally or distally relative to inner shaft 26, thereby respectively uncovering or covering the compartment with distal sheath 24. Outer shaft 22 may be made of a flexible material such as nylon 11 or nylon 12, and it may have a round braid construction (i.e., round cross-section fibers braided together) or flat braid construction (i.e., rectangular cross-section fibers braided together), for example.

The proximal end of inner shaft 26 may be connected in a substantially fixed relationship to an outer housing 30 of operating handle 20, and the proximal end of the outer shaft 22 may be affixed to a carriage assembly 40 that is slidable along a longitudinal axis of the handle housing, such that a user can selectively slide the outer shaft relative to the inner shaft by sliding the carriage assembly relative to the housing. Operating handle 20 may further include a hemostasis valve 28 having an internal gasket adapted to create a seal between inner shaft 26 and the proximal end of outer shaft 22.

As shown, handle housing 30 includes a top portion 30a and a bottom portion 30b. Top and bottom portions 30a and 30b may be individual components joined to one another as shown in FIG. 1B. Collectively, top and bottom portions 30a and 30b define an elongated space 34 in housing 30 in which carriage assembly 40 may travel. Optionally, top and bottom portions 30a and 30b may further form a substantially cylindrical boss 31 for accepting a clip, as will be described below. Elongated space 34 preferably permits carriage assembly 40 to travel a distance that is at least as long as the anticipated length of the prosthetic valve to be delivered (e.g., at least about 50 mm), such that distal sheath 24 can be fully retracted from around the prosthetic valve. Carriage assembly 40 includes a pair of carriage grips 42, each attached to a body portion 41. Although the carriage assembly 40 is shown in FIGS. 1A and 1B as having two carriage grips 42, that need not be the case.

Handle housing 30 further defines a pocket 37 that extends through top portion 30a and bottom portion 30b for receiving a deployment actuator 21. Pocket 37 is sized and shaped to receive deployment actuator 21 with minimal clearance, such that the location of deployment actuator remains substantially fixed relative to housing 30 as it is rotated. Deployment actuator 21 may be internally coupled to body portion 41 via a threaded shaft or other suitable connection such that rotation of the deployment actuator in one direction (either clockwise or counterclockwise) pulls the body portion 41 of carriage assembly 40 proximally through elongated space 34.

To use operating handle 20 to deploy a prosthetic valve that has been loaded into compartment 23 and covered by distal sheath 24, the user may rotate deployment actuator 21, causing carriage assembly 40 to slide proximally within elongated space 34 in housing 30. Because distal sheath 24 is affixed to outer shaft 22, which in turn is affixed to carriage assembly 40, and because inner shaft 26 is fixed to housing 30, sliding the carriage assembly proximally relative to the housing will retract the distal sheath proximally from compartment 23, thereby exposing and initiating deployment of the valve located therein.

Delivery device 10 may be used to implant a medical device such as a collapsible stent-supported prosthetic heart valve 100 having a stent 102 and a valve assembly 104 (FIG. 2). Prosthetic heart valve 100 is designed to replace a native tricuspid valve of a patient, such as a native aortic valve. It should be noted that while the devices disclosed herein are described predominantly in connection with their use to implant a prosthetic aortic valve and a stent having a shape as illustrated in FIG. 2, the valve could be a bicuspid or other valve, such as the mitral valve.

The expandable stent 102 of prosthetic heart valve 100 may be formed from biocompatible materials that are capable of self-expansion, such as, for example, shape memory alloys, such as the nickel-titanium alloy known as “nitinol,” or other suitable metals or polymers. Stent 102 extends in a length direction L1 from proximal or annulus end 110 to distal or aortic end 112, and includes annulus section 120 adjacent proximal end 110, transition section 121, and aortic section 122 adjacent distal end 112. Annulus section 120 has a relatively small cross-section in the expanded condition, while aortic section 122 has a relatively large cross-section in the expanded condition. Preferably, annulus section 120 is in the form of a cylinder having a substantially constant diameter along its length. Transition section 121 may taper outwardly from annulus section 120 to aortic section 122. Stent 102 may also have different shapes, such as a flared or conical annulus section, a less-bulbous aortic section, and the like, and a differently shaped transition section 121. Each of the sections of stent 102 includes a plurality of struts 130 forming cells 132 connected to one another in one or more annular rows around the stent. For example, as shown in FIG. 2, annulus section 120 may have two annular rows of complete cells 132 and aortic section 122 and transition section 121 may each have one or more annular rows of partial cells 132. Cells 132 in aortic section 122 may be larger than cells 132 in annulus section 120 to better enable prosthetic valve 100 to be positioned in the native valve annulus without the stent structure interfering with blood flow to the coronary arteries. Each of cells 132 has a length in length direction L1 of the stent and a width in a perpendicular direction W1.

Stent 102 may include one or more retaining elements 134 at distal end 112 thereof, retaining elements 134 being sized and shaped to cooperate with recesses 80 in retainer 25 of delivery device 10. The engagement of retaining elements 134 with portions of delivery device 10 helps maintain prosthetic heart valve 100 in assembled relationship with the delivery device, minimizes longitudinal movement of the prosthetic heart valve relative to the delivery device during unsheathing or resheathing procedures, and helps prevent rotation of the prosthetic heart valve relative to the delivery device as the delivery device is advanced to the target location and the heart valve deployed.

Valve assembly 104 of prosthetic heart valve 100 preferably is positioned in annulus section 120 of stent 102 and secured to the stent. Valve assembly 104 includes cuff 136 and a plurality of leaflets 138 which collectively function as a one-way valve by coapting with one another. As a prosthetic aortic valve, valve 100 has three leaflets 138.

Although cuff 136 is shown in FIG. 2 as being disposed on the luminal or inner surface of annulus section 120, it is contemplated that cuff 136 may be disposed on the abluminal or outer surface of annulus section 120 or may cover all or part of either or both of the luminal and abluminal surfaces. Both cuff 136 and leaflets 138 may be wholly or partly formed of any suitable biological material or polymer such as, for example, polytetrafluoroethylene (PTFE).

Leaflets 138 may be attached along their belly portions to cuff 136 or to stent 102, with the commissure between adjacent leaflets 138 being attached to a commissure feature 140. As can be seen in FIG. 2, each commissure feature 140 may lie at the intersection of four cells 132, two of the cells being adjacent one another in the same annular row, and the other two cells being in different annular rows and lying in end-to-end relationship. Preferably, commissure features 140 are positioned entirely within annulus section 120 or at the juncture of annulus section 120 and transition section 121. Commissure features 140 may include one or more eyelets which facilitate the suturing of the leaflet commissure to stent 102.

Prosthetic heart valve 100 may be used to replace a native aortic valve, a surgical heart valve or a heart valve that has undergone a surgical procedure. Prosthetic heart valve 100 may be delivered to the desired site (e.g., near the native aortic annulus) using any suitable delivery device, including delivery device 10 described above. During delivery, prosthetic heart valve 100 is disposed inside compartment 23 of delivery device 10 in the collapsed condition. The delivery device may be introduced into a patient using a transfemoral, transapical, transseptal or any other percutaneous approach. Once the delivery device has reached the target site, the user may deploy prosthetic heart valve 100 in the manner described above. Upon deployment, prosthetic heart valve 100 expands so that annulus section 120 is in secure engagement within the native aortic annulus. When prosthetic heart valve 100 is properly positioned inside the heart, it works as a one-way valve, allowing blood to flow from the left ventricle of the heart to the aorta, and preventing blood from flowing in the opposite direction.

FIG. 3 illustrates conventional polymer-based introducers 300 that utilize a folding technique to insert the sheath at a smaller diameter. Specifically, a portion of the introducer is folded upon itself in a first condition 300A for insertion and then unfurls to define a larger diameter of a second condition 300B. One disadvantage of a folded sheath is that blood may exit or leak through the folds when the sheath is expanded post insertion. Furthermore, it may be challenging to subsequently reduce the diameter of a folded sheath to a smaller diameter when the sheath is withdrawn after use. Thus, it would be desirable to provide a sheath that radially expands and collapses, which will minimize the friction against the vessel wall. Additionally, conventional sheaths may not have the ability to expand enough to enable large bore catheter to pass through. Regular expandable sheath might be capable of expansion by a maximum rate, whereas higher expansion rates might be desirable. Conventional sheath may also not be large enough to accommodate larger transcatheter mitral valve replacement and transcatheter tricuspid valve replacement devices without defeating the purpose of having a small introducer sheath to minimize vessel trauma.

An introducer sheath may be useful to provide access into a vessel to allow for advancing a delivery device to a target location in patients whose native anatomy has calcified legions or may be compromised due to degeneration of the vessel. Generally, the target is just beyond the femoral bifurcation point as to reduce the risk of tearing or lacerating the vessel during insertion of the larger delivery system profile. Specifically, an incision may be formed in the patient's body and the introducer sheath may be placed through the incision to provide a passageway for advancing a medical device into the patient's body. It has been found that larger introducers risk traumatizing body tissue and that the risk of trauma increases with time. Thus, it is desirable to have a small introducer sheath that locally expands as necessary. Although introducer sheaths are described below in connection with the delivery of a prosthetic heart valve into a patient, it will be understood that the concepts described may be useful for any interventional procedure in which an apparatus, such as a medical device or instrument, is passed through an introducer sheath for delivery, implantation or surgical procedures, such as other cardiac repair procedures, balloon angioplasty, laparoscopic surgical procedures, peripheral interventional procedures, and the like. In some examples, an introducer sheath that can be inserted at a small diameter (<18 F) and expand to the required diameter of a therapeutic or diagnostic device may reduce trauma to the vessel and the connecting tissue. It will be understood that arterial and venous access requirements for the devices may differ with respect to the desired diameter and expansion.

Moreover, the principles described herein may be applicable to other applications such as percutaneous ventricular assist devices (pVAD), leadless pacemakers, or any other large bore catheter inserted into a vessel of a patient where vascular access is deemed an operative risk (e.g., difficult to navigate iliac, jugular vein, small vessels, etc.). The present disclosure describes a sheath that may be introduced at a small outer diameter and can expand to accept larger devices, the sheath interaction against the native anatomy limiting circumferential fluid leak, and minimizing trauma of the vessel and connecting tissue. In some examples, a sheath according to the present disclosure is capable of accessing the vasculature approximately at an angle of 20-45 degrees (e.g., 30 degrees) with respect to the patient's back. In some examples, a sheath utilizes an access point that is just distal to the profunda and superficial artery bifurcation point. The sheath may navigate through the vasculature to beyond the main arterial bifurcation. The sheath may also be used for venous applications as well. Additionally, other access points (e.g., radial access, jugular access, etc.) may be possible. The sheath may be able to navigate tortuous patient anatomy, including potentially calcified vasculature. In some examples, a sheath may have appropriate bending stiffness to be trackable and insertable, while conforming to how the artery may naturally curve throughout the body.

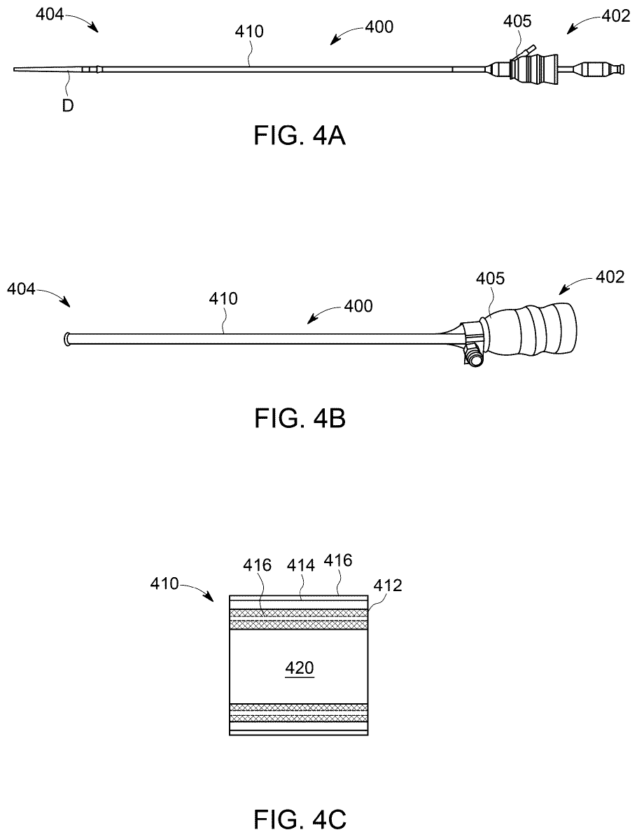

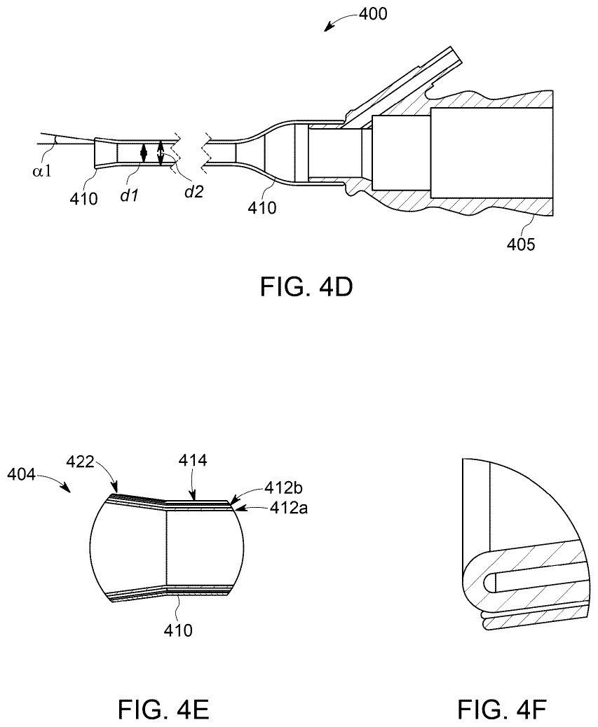

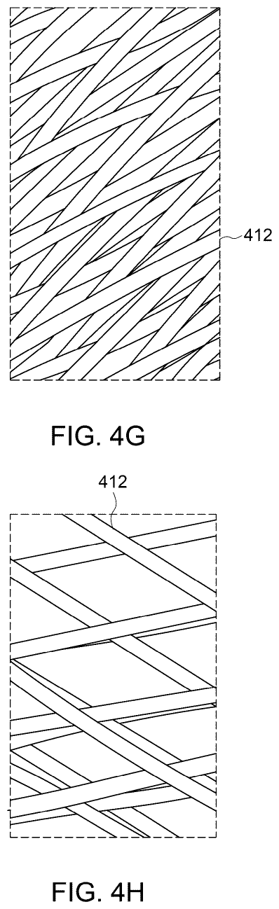

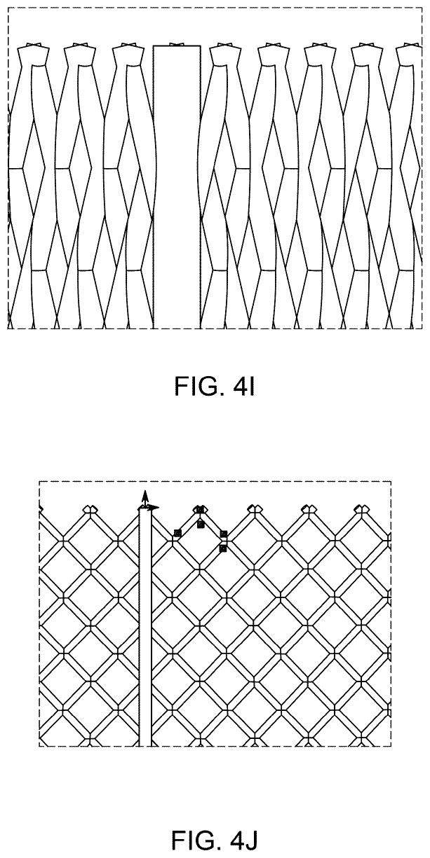

FIG. 4A-B illustrates one example of an introducer sheath 400 that generally extends between a proximal end 402 and a distal end 404. Introducer sheath 400 may be introduced at a small outer diameter and expand to accept larger devices. Due to the introducer sheath construction, the risk of leakage is reduced compared to conventional devices. Introducer sheath 400 may include a hub 405 disposed adjacent the proximal end, and a body 410 that extends from the hub adjacent proximal end 402 to the distal end 404. In FIG. 4A, a dilator “D” is shown disposed within body 410 of the introducer. As shown in the schematic of FIG. 4C, body 410 may comprise an inner braided material 412 (e.g., NiTi or “nitinol” braid) and an outer elastomeric material 414 at least partially covering the braided material 412, the body defining a central lumen 420 for receiving instruments, devices, dilators, prosthetic implants and the like. Additional support structures (not shown) may be added to the body 410. While conventional sheaths include an inner polymeric liner, body 410 may include a braided material 412 having an inner surface capable of receiving instruments, devices and prosthetic implants therethrough. In some examples, the braided material 412 includes NiTi with a high surface finish that is not prone to catching on material passing along its surface. In some examples, braided material 412 may include NiTi braid having round or flat wire. Optionally, additional polymeric layer(s) 416 may be added over outer elastomeric material 414 and/or within braided material 412. This may yield additional benefits such, as for example, increased column strength. In some examples, the additional polymeric layer(s) 416 may comprise Pebax. The additional polymeric layer 416 may be the outermost layer, the innermost layer, or may sandwich elastomeric material 414 and braided material 412. The additional polymeric layer(s) 416 may also include a double layer disposed on the outside the elastomeric material 414, or within braided material 412. In some examples, elastomeric material 414 and/or additional polymeric layer(s) 416 may be integrated (e.g., dipped, sprayed, extruded, etc.) over braided material 412 to cover the entire braided material 412 or into a pattern (e.g., candy-cane striped extrusions of different durometers). In some examples, a Heparinized saline may be utilized during a procedure to ensure no thrombus are created within braided material 412.

Turning to FIGS. 4D-E, certain details of the introducer sheath 400 will be described. As shown in FIG. 4D, body 410 may be coupled to the hub 405 adjacent the proximal end. In some examples, body 410 is flared at the proximal end and couples to the outer diameter of hub 405 to create a smooth transition. In some examples, body 410 may be between 160 and 900 mm in length, or between 300 and 400 mm in length (e.g., between 350 mm and 400 mm in length, or approximately 360 mm in length). Elastomeric material 414 may be bonded, heat-bonded, or flowed onto braided material 412 along the length of the body 410. In some examples, the internal diameter d1 of the braided material is between 2 mm and 8 mm (e.g., 4 mm), and in some examples, the outer diameter d2 is between 2 mm and 8 (e.g., 4.97 mm).

Turning to FIG. 4E, in some embodiments, a single length of braided material is used, which is double the final catheter length. For example, a braided material of approximately 726 mm in length may be used to create a body length of 363 mm. Thus, the braided material 412 may comprises two or more layers including an inner braided layer 412a and an outer braided layer 412b, the two layers being at least partially covered by elastomeric material 414. A flared distal end 422 may also be formed, and the flare may have an angle α1 of between 1 and 45 degrees (e.g., 5 degrees, 7 degrees, 10 degrees or 15 degrees) defined with respect to the longitudinal axis of the body 410 to facilitate instrument withdrawal. The braided material may transition between a collapsed condition and an expanded condition. In some examples, the PIC count (i.e., the per inches wire crosses, or wire cross density), density and lead may be high enough not to interfere with the devices passing through the inner diameter of the sheath. The braided material may be heat-set in the collapsed condition (i.e., at the unexpanded diameter) with an appropriate tip shape applied to not interfere with any of the passed through devices in case device withdrawal is desired. In some examples, a “roll” may be applied to the distal tip, as seen in FIG. 4F, so that the tip does not break under an acute bend. This tip configuration may be advantageous because the NiTi freetails of the braid are not exposed, which may otherwise limit the expansion due to the need to manage the freetails.

After the doubling over and the heat-setting process, the braided material is reduced to about half its original single-layer length. In some examples, the thickness of the wire strands of the braided material may be between 0.001″ to 0.008″ depending on the stiffness required of the sheath. This results in a relatively thin wall thickness of between 0.002″ and 0.016″ (e.g., 0.015″) and a thickness of between 0.007″ and 0.030″ with a polymeric coating.

In some examples, a tubular braided material 412 may be configured and arranged to avoid “finger trapping” when a tool or instrument is pushed through its lumen. Without being bound by any particular theory, it is believed that one or more of a combination of the PIC count, the angle of the braid, the materials of the braid and/or the specific process of heat setting may contribute to a reduction in finger-trapping. For example, starting with a larger diameter and a high PIC count may yield a relatively high resulting lead angle of braid after heat setting. As shown in FIG. 4G, when collapsed the wires of the braided material overlap and display a relatively high density within a certain region, and the angle between wires is relatively acute (e.g., less than 20 degrees, less than 15 degrees, or less than 10 degrees). Conversely, as shown in FIG. 4H, the expanded PIC angle is larger (e.g., between 30 and 60 degrees, or approximately 45 degrees as shown), and the density of wires is relatively lower in the same region. As shown in certain models, the angle between wires of a 144 braided material increased from between 20-40 degrees, between 25-30 degrees or 27 degrees (as shown in FIG. 4I) in the collapsed condition when the braided material forms a tube with a 6 mm outer diameter, to between 80 and 90 degrees, or 84 degrees (FIG. 4J) in the expanded condition when the braided material forms a tube with 25 mm outer diameter.

As shown in FIG. 4C, elastomeric material 414 may be arranged as a single layer or double layer. In some examples, the elastomeric material 414 includes a single layer at least partially covering the outer diameter on the braided material 412. In some examples, elastomeric material 414 comprises a double layer, which includes a first layer on the outer diameter of the braided material and a second elastomeric material disposed in between braid layers. Embodiments that include multiple elastomeric materials may allow the melting of the two materials for superior adhesion to the braided material. In some examples, wall thicknesses of the elastomer may be as thin as 0.001″ and as thick as 0.015″, depending on the desired force required to open the elastomer. In some examples, the elastomeric material 414 comprises chronoprene, Pebax (e.g., 25D Pebax), silicone, Tecothane, Hydrothane, Chronoflex, ePTFE, bicarbonate and other similar materials or combinations thereof. These elastomers can also be strategically striped, hydrophilic coated, and controlled via material design parameters. In some embodiments, the term “elastomeric” is used to describe any polymer capable of forming a tube that is expandable to at least twice its diameter with acceptable forces and returning to its initial state absent external forces.

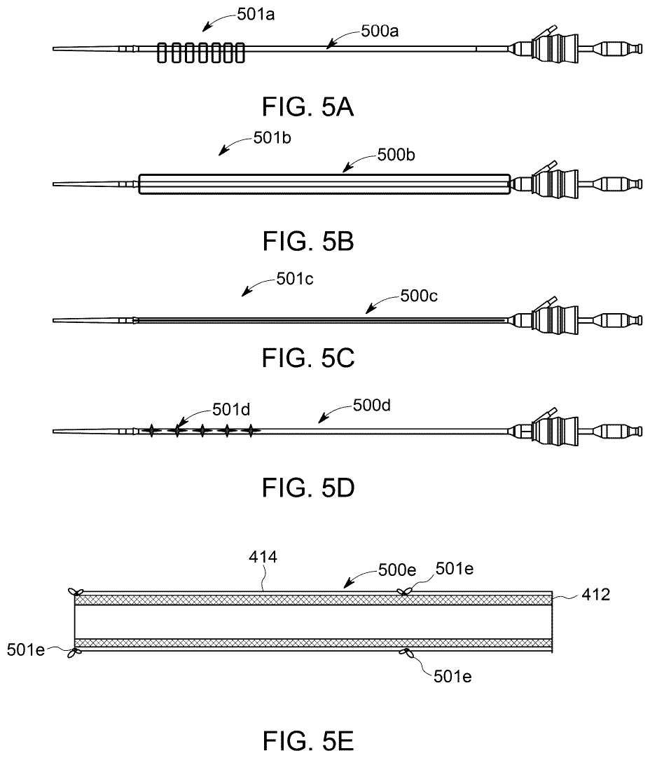

FIGS. 5A-D illustrate several examples of attaching the elastomeric material to the braided material. In some examples, the elastomeric material may be coupled, or attached, to the braided material in a way to allow appropriate expansion and/or reduction in diameter as the introducer sheath transitions from the collapsed condition to the expanded condition and vice versa. In the example shown in FIG. 5A, an introducer sheath 500a may include an elastomeric material 414 coupled to the braided material 412 at segmented reflow attachment regions or bands 501a that are axially-spaced apart from one another. The bands 501a may be disposed adjacent only the distal end, only the proximal end or throughout the body. In some examples, the bands 501a are axially-spaced apart from one another by 1-5 centimeters. In another example shown in FIG. 5B, an introducer sheath 500b may include an elastomeric material 414 coupled to the braided material 412 with a complete reflow of the elastomeric material along the entire length of the body. In FIG. 5C, introducer sheath 500c includes a spine reflow attachment 501c along the length of the body. In some examples, a spine reflow includes a thin axial attachment approximately 1 mm wide. Sheath 500c may include a single spine reflow attachment 501c or multiple spine reflow attachments (e.g., two, three, four or more), circumferentially disposed along the body and spaced from one another (e.g., separated by a constant radial distance from one another). Finally, in FIG. 5D, dimplings or spot welds 501d are used to couple the elastomeric material to the braided material. In this examples, five welds 501d are shown, axially spaced from one another adjacent the distal end. It will be understood that any number of welds may be disposed adjacent the proximal and/or distal ends, and that the welds may be disposed along a single side of the sheath 500d, or radially disposed about the body (e.g., two or more welds circumferentially spaced from on another at each axial extent). Any of these configurations may be used to couple the elastomeric material to the braided material and to reduce or eliminate bunching of the elastomeric material during insertion. Optionally, an outer hydrophilic coating may be applied to the elastomeric material to reduce the friction between the elastomer and the native tissue. Hydrophilic elastomers may also be used as the elastomeric material.

Instead of, or in addition to, reflowing materials, suture attachment of the elastomeric material to the braided material may also be possible (FIG. 5E). In some examples, sutures 501e may be disposed at the distal end of an introducer sheath 500e to couple the layers together. In some examples, sutures 501e may also be disposed at the proximal end, or at any point between the proximal end and the distal end. Sutures 501e may be circumferentially disposed about the body with two, three, four or more stitches being radially spaced apart from one another. Sutures 510e may also include between one and ten stitches disposed around the diameter at various axial positions.

Variations of the foregoing embodiments are possible. For example, an introducer sheath may be formed using a combination of elements described above. Additionally, in some examples, one or more elements of the introducer sheath may be capable of changing stiffness upon the application of heat, cooling, electricity or other external stimuli, such as pneumatics (i.e., pressure). For example, an introducer sheath may be relatively flexible and stiffen up once the external stimuli is applied. This may allow the introducer sheath to be trackable and insertable, but not too stiff where it cannot conform to how the artery may naturally curve throughout the body. The application of stimuli at certain points in the procedure may allow the clinician to alternate between a relatively stiff configuration and a relatively flexible configuration at different steps in the procedure. The introducer sheath may also include one or more radiopaque elements disposed on the circumference along one or more axial positions to allow the physician to take an image to see if a consistent tubular shape is defined within the vasculature by the introducer sheath. This may, for example, be useful for locating calcium deposits or to visualize post-dilator deployment to ensure that the introducer sheath is not buckled or deformed prior to insertion of a tool or implant.

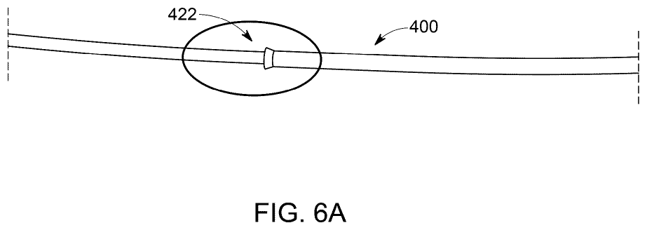

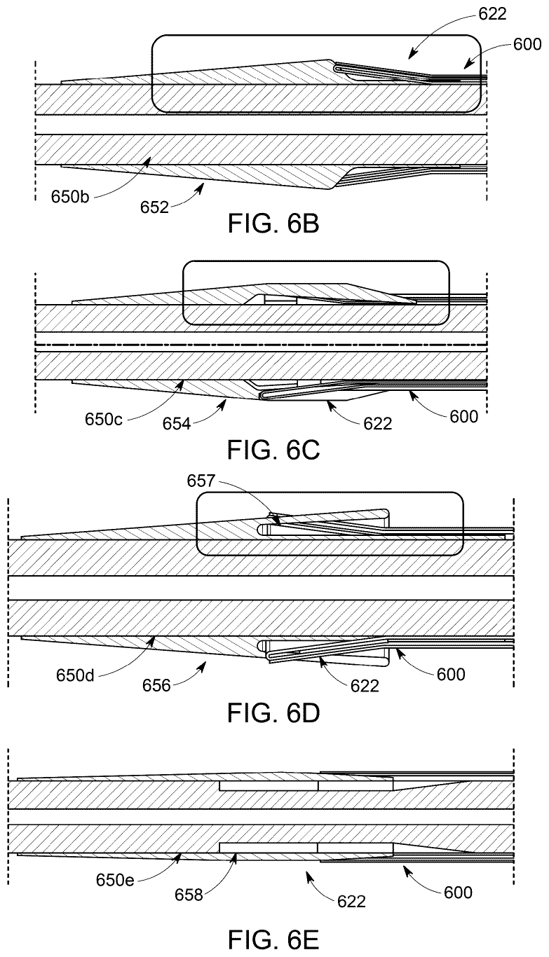

As previously noted, an introducer sheath 400 may include a flared distal end 422 defining an angle of between 5 and 45 degrees (e.g., 7 degrees, 10 degrees or 15 degrees) to facilitate instrument withdrawal through the distal end of the sheath. In some examples, a flared distal end may reduce the potential for device prolapse (FIG. 6A). Without being bound by any particular theory, it is believed that the distal flare inflects the tip of the sheath away from the center of the lumen. This tipping may allow an interrupted profile device (being passed through the introducer sheath) that may contain fabric, tissue, metal, or polymer features to be withdrawn without “catching” at the tip. The flare may provide a “ramp” for the introducer sheath to accommodate nonconformities in the shaft or delivery system being withdrawn. In some examples, during insertion of the expandable introducer sheath, the distal end of the introducer sheath may include a smooth transition from the sheath to the dilator to avoid damaging the vessel. In some examples, it may be desirable to design a dilator which will allow a smooth transition and/or to retain the flared distal end. Thus, in addition to the flared distal end, certain complementary features may be included on the dilator. Once inside the vessel the flared distal end is not expected to negatively interact with the anatomy. In some embodiments, features on the dilator may shield or retain the flared distal end of the sheath from the access tissue.

Several possible dilator features are shown in FIGS. 6B-9C to complement, retain or shield the flared distal end 622 of an introducer sheath 600. In FIG. 6B, dilator 650b includes an angled butt 652 disposed opposite flared distal end 622, the angled butt 652 having a maximum outer diameter closer to the flared distal end 622, and a linearly (or non-linearly) decreasing outer diameter distally thereof. In FIG. 6C, dilator 650c includes an encapsulating segment 654 configured to slide and receive flared distal end 622 therein, encapsulating segment 654 being substantially domed to house the flared distal end 622. In FIG. 6D, dilator 650d includes a receiving segment 656 including a channel 657 for accepting the flared distal end 622 therein, the channel 657 being substantially equal in length to the flared distal end 622. In FIG. 6E, dilator 650e includes a radially undercut slot 658 for accepting the flared distal end 622 therein, the radially undercut slot 658 being defined radially inward of the outer diameter of dilator 650d.

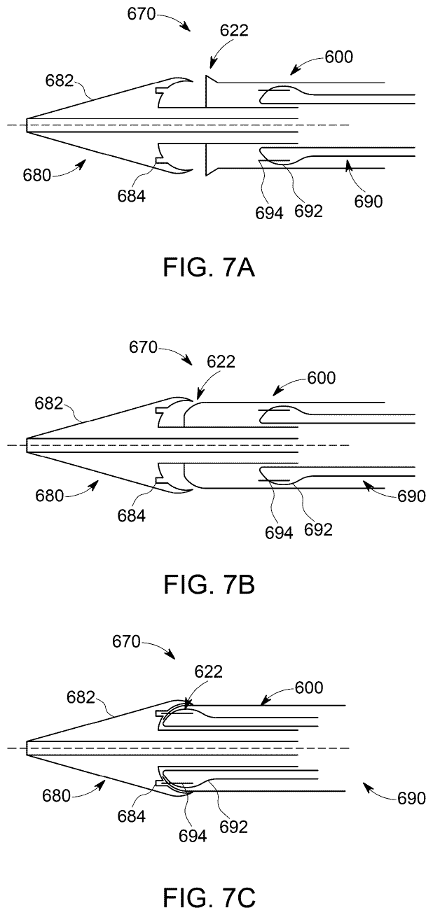

Another embodiment is shown in FIGS. 7A-C, which illustrate various stages of a two-part dilator. Specifically, introducer sheath 600 is shown with a flared distal end 622, and a dilator 670 having a distal segment 680 and a proximal segment 690, the distal segment and the proximal segment being coupleable with one another. Distal segment 680 may include a leading nosecone tip 682 and one or more recesses 684, and proximal segment 690 may include a bulbous portion 692 and one or more locking pins 694. In some examples, the number of recesses 684 and the number of locking pins 694 are equal, and the two elements are aligned or alignable with one another. As shown in FIG. 7A, proximal segment 690 may be initially disposed within introducer sheath 600. As distal segment 680 and proximal segment 690 are brought together for engagement, flared distal end 622 may be inwardly biased and sandwiched between the two elements (FIG. 7B). The operator may continue to bring distal segment 680 and proximal segment 690 into engagement until the one or more recesses 684 receive the one or more locking pins 694, and the locking pins 694 are secured within the recesses (FIG. 7C). In some examples, the operator may “assemble” the dilator and the introducer sheath. Alternatively, the two components may be “preassembled” with the operator only having to insert the assembled sheath into the vessel. In this condition, the flared distal end 622 is secured between distal segment 680 and proximal segment 690. Specifically, flared distal end 622 may overlie bulbous portion 692 and be secured within distal segment 680. In use, dilator 670 may be engaged with introducer sheath 600 as shown in FIG. 7C, and delivered to the target position. Distal segment 680 and proximal segment 690 may begin to be disengaged from one another (e.g., by pushing distal segment 680 forward and keeping proximal segment 690 stationary, or by pulling proximal segment 690 backward while keeping distal segment 680 stationary, or some combination thereof). Disengagement of distal segment 680 from proximal segment 690, may release flared distal end 622 of introducer sheath 600 so that the flared distal end 622 returns to its resting, radially-opened condition.

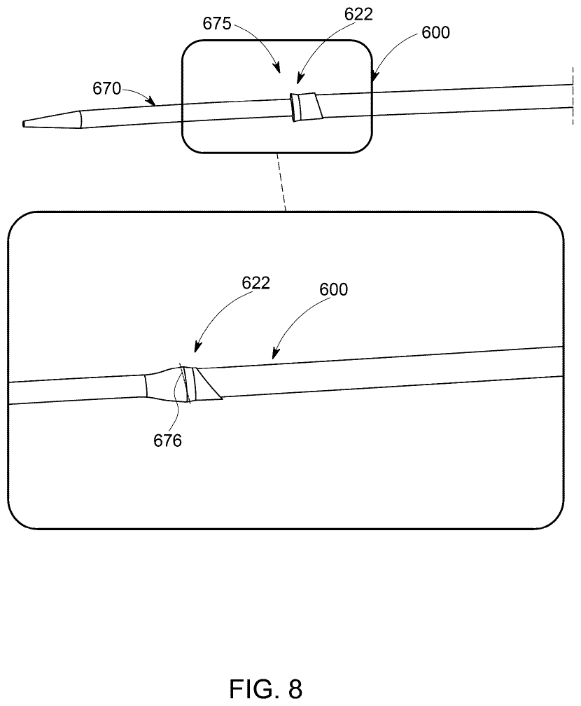

An elastomer may also be used to provide a smooth transition between a dilator and an introducer sheath, and to at least partially retain the flared distal end of an introducer sheath closer to the dilator. In FIG. 8, an elastomer 675 is bonded to the surface of the dilator 670 and extends proximally beyond the flare distal end 622 of the introducer sheath 600. Just proximal to the flared tip end 622 of introducer sheath 600, the elastomer 675 may include a horizontally scored or perforated line 676 to allow a controlled tearing of the elastomer so that it can be removed once the dilator 670 is extended a set distance distal to the tip of the introducer sheath 600. In some examples, the elastomer 675 may be released by pushing the dilator distally, thereby deploying the flared distal end of the introducer sheath.

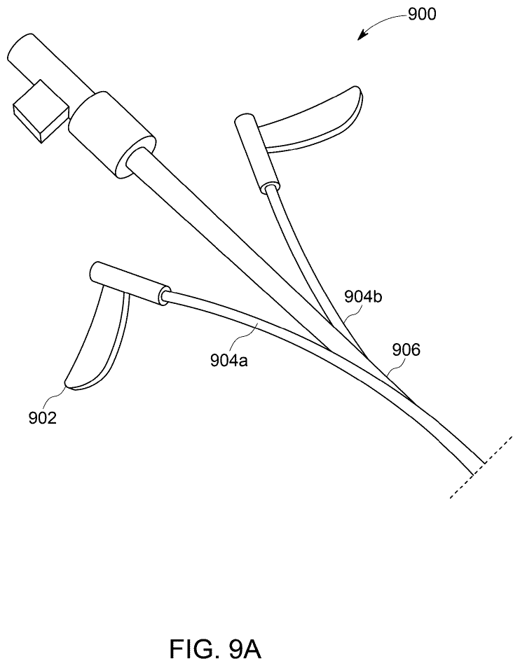

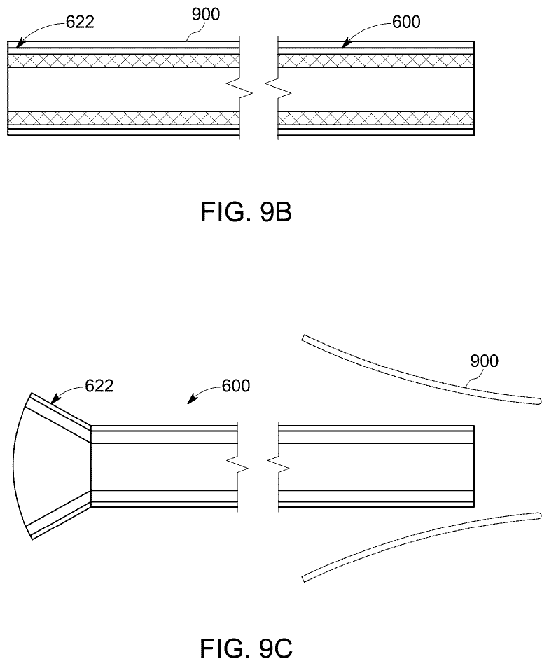

Because the outer diameter of a braided introducer sheath is relatively small, it may be possible to add a “cover” sheath on the outer diameter to restrict full expansion of the distal flared end and allow a seamless transition with the dilator. A split sheath 900 (or “peel-away sheath”) is shown in FIG. 9A, which includes two pull tabs 902 and a cylindrical tube divided into two portions 904a,904b. Split sheath 900 may include a longitudinal scoring 906 that allows the two portions 904a,904b to separate from one another when the pull tabs 902 are pulled apart. As shown in FIG. 9B, a split sheath 900 may cover an introducer sheath 600 having a flared distal end 622, and retain the flared distal end 622 in a substantially axial configuration. When tabs 902 are pulled apart, the split sheath 900 may separate into two portions 904a,904b and be removed, thereby releasing the flared distal end 622 and allowing it to extend radially outward as shown in FIG. 9C.

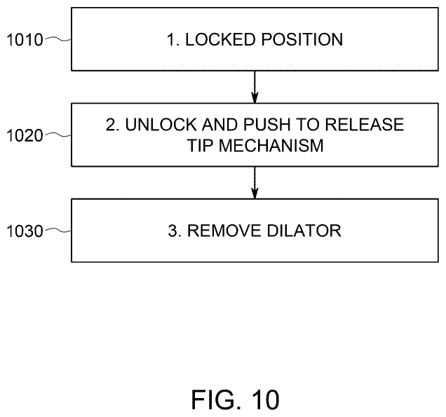

In some embodiments, a dilator and introducer sheath hub may lock together to give column strength to the braided material and the elastomer. In some examples, both the braid material and elastomeric material are not strong when compressed axially. Once the tip of the sheath is starting to enter the surrounding soft tissue towards the vascular access, the physician should be able to push on the hub without prolapse of the device. To reduce the risk of prolapse, the dilator may lock into, or with, the hub. In some examples, three positions are used, as ordered in the procedure flow chart of FIG. 10. In this example, the hub and dilator begin in the locked position in step 1010, then the dilator is unlocked from the hub and pushed to release the tip mechanism in step 1020, followed by dilator removal in step 1030.

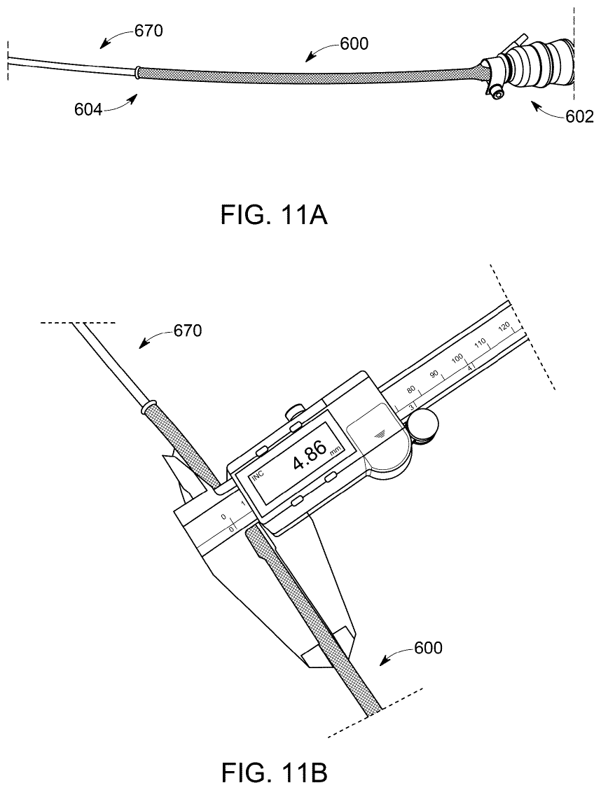

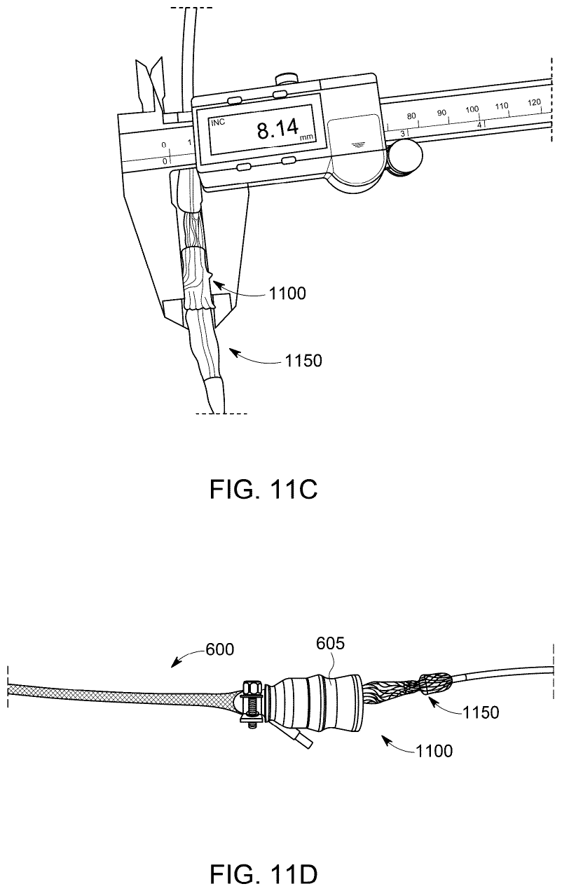

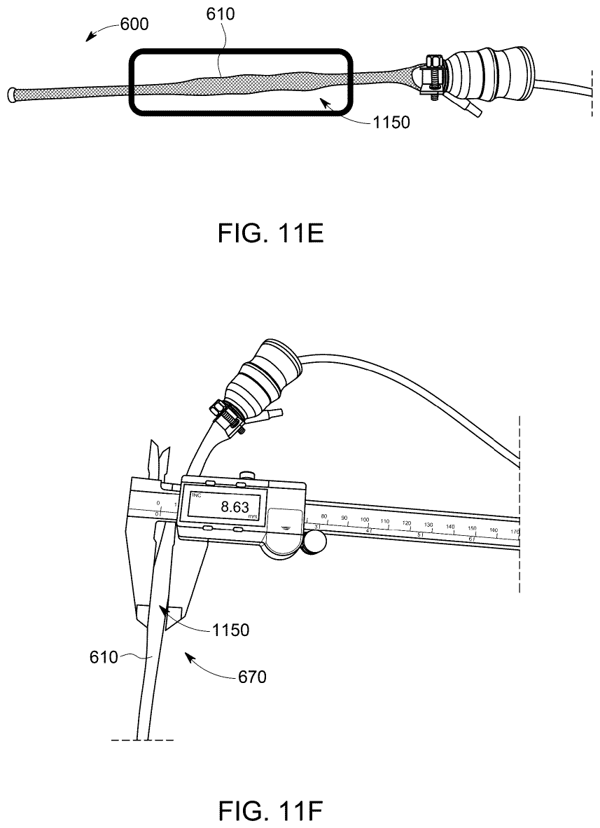

FIGS. 11A-F illustrate the use of an introducer sheath 600 that extends between a proximal end 602 adjacent a hub, and a distal end 604. In the example shown in FIG. 11A, the introducer sheath 600 includes a dual layer of braided material and a single elastomeric material. A 12 French (4 mm) dilator 670 is shown extending through introducer sheath 600. The outer diameter of introducer sheath 600 is shown to be 4.86 mm or approximately 15 French with the dilator 670 being disposed therein. In FIG. 11C, a collapsed prosthetic heart valve 1100 is being shown within a delivery catheter 1150, and this assembly has an outer diameter of approximately 8.14 mm or approximately 24 French. This same assembly of a prosthetic heart valve 1100 housed within a delivery catheter 1150 is being introduced through hub 605 of introducer sheath 600 in FIG. 11D, and is within the introducer sheath body 610 in FIG. 11E. As shown in FIG. 11F, the outer diameter of the introducer sheath 600 with prosthetic heart valve 1100 and delivery catheter 1150 being disposed therein is approximately 8.63 mm or approximately 26 French.

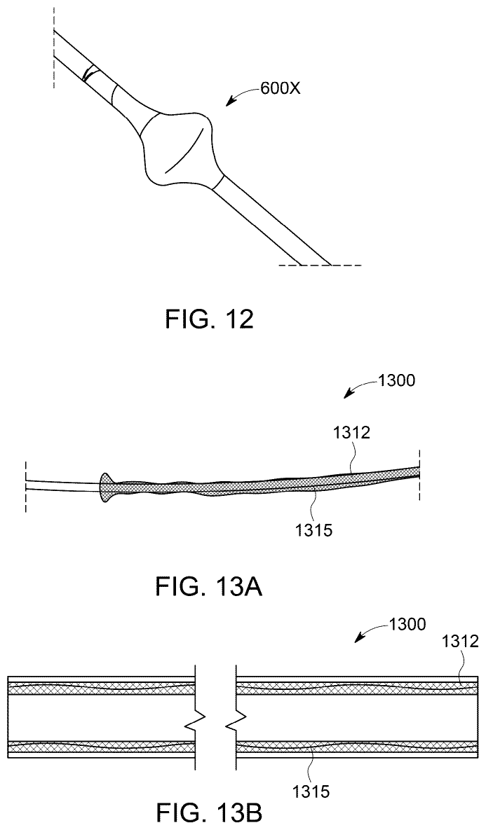

When introducer sheath is inserted into tissue, a column load will be applied to the sheath. While the tipping is designed to carry the brunt of the load, the braided material and elastomeric material may expect load when passing through tissue. If the load is too high, there is a risk of prolapsing the sheath with the braid expanding under compression, as seen in FIG. 12 which shows an introducer sheath 600X at maximum expansion. To prevent or reduce the risk of prolapse, an introducer sheath 1300 may include a braided material 1312 and buttressing axial wires 1315 (e.g., NiTi wires). Axial wires 1315 may be interwoven into the braided material 1312, or disposed between two layers of braided material 1312 and attached distally at the hub. Axial wires 1315 may be generally parallel with a longitudinal axis of the body, as shown in FIGS. 13A-B. In some examples, axial wires 1315 are serpentine. The elastomeric material (not shown) may then be applied over the combination of the axial wires and the braided material. In this example, when force is applied to the braided material 1312, it is limited in its expansion by the strength of the axial wires 1315. Optionally, the axial wires 1315 may be co-braided and double backed in the same nature as the braid is described above.

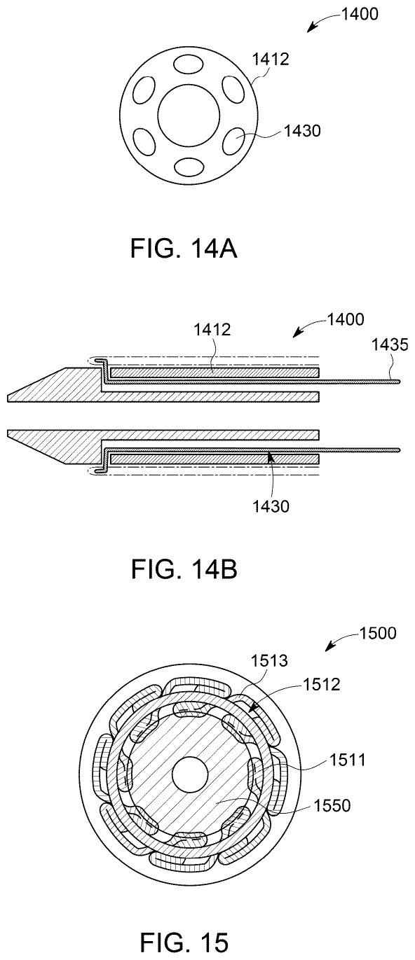

In some embodiments, it may be useful to tension the introducer sheath (e.g., the braided material) against a dilator to prevent prolapse of the introducer sheath during insertion. This may be particularly advantageous for expected higher insertion forces due to calcification of the vessel restricting the percutaneous site. In some examples, an introducer sheath 1400 includes a braided material 1412 having a plurality of peripheral lumens 1430 as shown in FIG. 14A. In this example, introducer sheath 1400 included a braided material 1412 with six peripheral lumens. It will be understood that one, two, three, four, five, six, seven or eight or more peripheral lumens are possible. Introducer sheath 1400 may also include a stabilizing wire 1435 corresponding to each peripheral lumen. The stabilizing wire(s) 1435 may put the braided material in tension and prevent pullback during insertion. In some examples, stabilizing wires(s) 1435 may be pulled from the proximal end to release the braided material and relieve the tension on the braided material.

In some embodiments, non-elastic elements (e.g., Polyethylene, PET, polyesters, Polyurethane, Pebax, nylon et.) may be used to create an expandable introducer sheath. Additionally, pleating and/or folding may be used to reduce the diameter of an introducer sheath. It will be understood that non-elastic elements and/or pleating and/or folding techniques may be used with any combination of the embodiments described herein. FIG. 15 illustrates on embodiment of an introducer sheath 1500 that utilizes such these techniques. As shown, introducer sheath 1500 may generally include three layers from the inside to the outside, as follows. First, introducer sheath 1500 may include a braided material 1512 or a lasercut tube (e.g., NiTi lasercut tube). If a lasercut tube is used, a folded inner liner 1511 may be disposed therein. In some examples, the folded inner liner 1511 is pleated and folded as shown. Suitable materials for the folded inner liner 1511 may include PTFE, Pebax, polyethylene, etc. A folded outer jacket 1513 may be disposed on the outside of the braided material 1512 or lasercut tube. In some examples, folded outer jacket 1513 is lubricious and safe enough to abut a vessel. The folded outer jacket 1513 may be pleated and folded, and may comprise PTFE, Pebax, polyethylene, etc. Finally, a dilator 1550 is shown being disposed inside the folded inner liner 1511, and the dilator 1550 may comprise high-density polyethylene, low-density polyethylene, BaSO₄ or other similar materials.

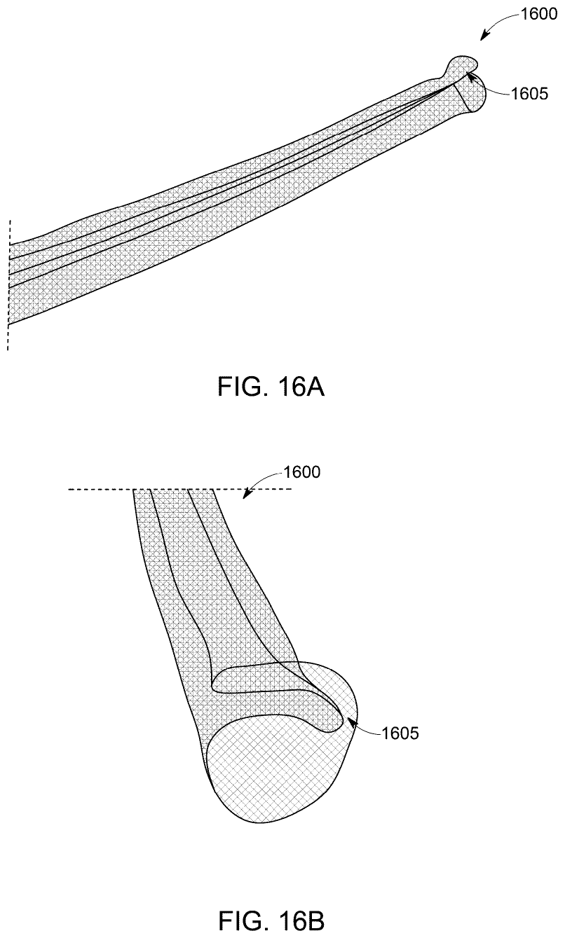

In the example above, the braided material 1512 is expandable and is shown as being unfolded. In some, the pleating and/or folding may be applied to the braided material in addition to, or instead of, the inner liner and the outer jacket. This may be advantageous if the braided material itself creates too much force for a device to easily expand the sheath during use. As shown in FIGS. 16A-B, the braided material 1600 may be formed into a tube, heat-set in its expanded form, then axially folded along pleat 1605, once or multiple times depending on the application to form a tube with a smaller diameter. Without being bound by any particular theory, it is believed that the force to expand a pleated/folded braided material may be far less than the elastomeric concepts described above. With this embodiment, one or more clastic polymeric jackets may be used, or one or more pleated and folded non-elastic jackets, or combinations thereof.

It will be understood that various modifications may be made to the disclosed embodiments without departing from the spirit of the disclosure. For example, an introducer sheath may be used to introduce a delivery device into the heart for prosthetic heart valve replacement, or may be used to introduce devices for valve repair at any of the heart valves (e.g., aortic valve, mitral valve, pulmonary valve, tricuspid valve). Additionally, an introducer sheath may be used to deliver instruments to repair other structures in the heart, such as the chordae tendineae, papillary muscles and the like. Introducer sheaths may also be used to deliver embolism prevention devices and stents, grafts and other cardiovascular devices into a patient, to introduce devices and instruments for other cardiac repair, to introduce any other medical instruments or devices into a patient's body in applications other than cardiovascular applications, and to access any bodily location where temporarily affixing a sheath within body tissue is useful.

It will be appreciated that the various dependent claims and the features set forth therein can be combined in different ways than presented in the initial claims. It will also be appreciated that the features described in connection with individual embodiments may be shared with others of the described embodiments.

1. An introducer sheath extending from a proximal end to a distal end, comprising:
a hub disposed at the proximal end; and
a body coupled to the hub and extending between the proximal end and the distal end, the body defining a lumen and having a collapsed condition and an expanded condition, the body having a braided material and an elastomeric material covering the braided material, the body having a flared distal end.

2. The introducer sheath of claim 1, wherein the braided material comprises a nitinol braid.

3. The introducer sheath of claim 1, wherein the braided material comprises at least one of flat wires and round wires of a thickness between 0.001″ to 0.008″.

4. The introducer sheath of claim 1, wherein the braided material comprises a single length of braided tubing that is doubled over to form two layers.

5. The introducer sheath of claim 4, wherein the braided tubing includes freetails that are disposed at the proximal end when the braided tubing is doubled over.

6. The introducer sheath of claim 1, wherein the braided material is heat-set in the collapsed condition.

7. The introducer sheath of claim 1, wherein the flared distal end defines an angle of between 5 and 45 degrees with a longitudinal axis of the body.

8. The introducer sheath of claim 1, wherein the elastomeric material is coupled to the braided material at segmented reflow attachment regions that are spaced from one another.

9. The introducer sheath of claim 1, wherein the elastomeric material is coupled to the braided material via a reflow of the elastomeric material along an entire length of the body.

10. The introducer sheath of claim 1, wherein the elastomeric material is coupled to the braided material via a reflow of the elastomeric material along an axial spine.

11. The introducer sheath of claim 1, wherein the elastomeric material is coupled to the braided material via reflowed spot welds of the elastomeric material.

12. The introducer sheath of claim 1, wherein the elastomeric material is coupled to the braided material via one or more sutures.

13. The introducer sheath of claim 1, further comprising at least one axial buttressing wire interwoven with the braided material and extending parallel with a longitudinal axis of the body.

14. The introducer sheath of claim 1, wherein the braided material defines at least one peripheral lumen, and further comprising at least one stabilizing wire disposed within the at least one peripheral lumen and removable therefrom.

15. A system comprising:
the introducer sheath of claim 1; and
a dilator having a flared distal end retaining feature.

16. The system of claim 15, wherein the dilator includes a retaining feature that comprises at least one of (i) an angled butt with a maximum outer diameter closer to the flared distal end, (ii) an encapsulating segment configured to slide over and receive the flared distal end therein, (iii) a channel for accepting the flared distal end, and (iv) a radially undercut slot defined radially inward of an outer diameter of the dilator.

17. The system of claim 15, wherein the dilator includes a distal segment and a proximal segment coupleable to the distal segment, the distal segment and the proximal segment being configured and arranged to sandwich the flared distal end of the body therebetween.

18. The system of claim 17, wherein the proximal segment comprises a bulbous region, wherein the distal segment comprises one or more recesses, and the proximal segment comprises one or more locking pins that are coupleable to the one or more recesses.

19. The system of claim 15, wherein the flared distal end retaining feature comprises a split sheath disposed over the flared distal end of the body.

20. An introducer sheath extending from a proximal end to a distal end, comprising:
a hub disposed at the proximal end; and
a body having a lumen and extending between the proximal end and the distal end, the body having a collapsed condition and an expanded condition, the body having a double layer of a braided material and an elastomeric material covering the braided material.
